(19) United States
(12) Patent Application Publication
(10) Pub. No.: US 2020/0381470 A1
(43) Pub. Date: Dec. 3, 2020

(54) IMAGE CAPTURE DEVICE

(71) Applicant: Gingy Technology Inc., Hsinchu City (TW)

(72) Inventors: Mon-Nan Ho, Hsinchu City (TW); Jen-Chieh Wu, Hsinchu City (TW); Hao-Xiang Lin, Hsinchu City (TW)

(73) Assignee: Gingy Technology Inc., Hsinchu City (TW)

(21) Appl. No.: 16/996,883

(22) Filed: Aug. 18, 2020

Related U.S. Application Data
(63) Continuation-in-part of application No. 16/571,207, filed on Sep. 16, 2019.
Application No. 16/571,207 is a continuation-in-part of application No. 16/008,037, filed on Jun. 14, 2018, now Pat. No. 10,460,188.
Application No. 16/008,037 is a continuation-in-part of application No. 15/713,693, filed on Sep. 24, 2017, now Pat. No. 10,043,847.
Application No. 15/713,693 is a continuation-in-part of application No. 15/208,619, filed on Jul. 13, 2016, now Pat. No. 9,977,947.
Application No. 15/208,619 is a continuation-in-part of application No. 14/835,130, filed on Aug. 25, 2015, now abandoned.
Application No. 14/835,130 is a continuation-in-part of application No. 14/978,237, filed on Dec. 22, 2015, now Pat. No. 9,770,199.
Application No. 16/008,037 is a continuation-in-part of application No. 15/719,575, filed on Sep. 29, 2017, now Pat. No. 10,713,521.
Application No. 15/719,575 is a continuation-in-part of application No. 15/588,700, filed on May 8, 2017, now Pat. No. 10,455,129.
Application No. 15/719,575 is a continuation-in-part of application No. 15/399,704, filed on Jan. 5, 2017, now Pat. No. 10,356,296.
Application No. 16/008,037 is a continuation-in-part of application No. 15/942,386, filed on Mar. 30, 2018, now Pat. No. 10,122,899.
Application No. 15/942,386 is a continuation-in-part of application No. 15/826,711, filed on Nov. 30, 2017, now Pat. No. 9,964,436.
Application No. 15/826,711 is a continuation-in-part of application No. 15/239,842, filed on Aug. 18, 2016, now Pat. No. 10,049,256.
Application No. 16/008,037 is a continuation-in-part of application No. 15/976,883, filed on May 11, 2018, now Pat. No. 10,091,402.
Application No. 15/976,883 is a continuation-in-part of application No. 15/239,842, filed on Aug. 18, 2016, now Pat. No. 10,049,256.
Application No. 16/008,037 is a continuation-in-part of application No. 15/989,123, filed on May 24, 2018, now Pat. No. 10,181,069.
Application No. 15/989,123 is a continuation-in-part of application No. 15/239,842, filed on Aug. 18, 2016, now Pat. No. 10,049,256.
Application No. 16/008,037 is a continuation-in-part of application No. 15/956,875, filed on Apr. 19, 2018, now Pat. No. 10,713,465.
Application No. 15/956,875 is a continuation-in-part of application No. 15/588,700, filed on May 8, 2017, now Pat. No. 10,455,129.
Application No. 15/956,875 is a continuation-in-part of application No. 15/399,704, filed on Jan. 5, 2017, now Pat. No. 10,356,296.
Application No. 15/956,875 is a continuation-in-part of application No. 15/719,575, filed on Sep. 29, 2017, now Pat. No. 10,713,521.
Application No. 16/008,037 is a continuation-in-part of application No. 16/004,459, filed on Jun. 11, 2018, now abandoned.
Application No. 16/004,459 is a continuation-in-part of application No. 15/239,842, filed on Aug. 18, 2016, now Pat. No. 10,049,256.
(60) Provisional application No. 63/003,929, filed on Apr. 2, 2020.
(60) Provisional application No. 62/906,103, filed on Sep. 26, 2019.
(60) Provisional application No. 62/895,034, filed on Sep. 3, 2019.
(60) Provisional application No. 62/784,755, filed on Dec. 25, 2018.
(60) Provisional application No. 62/486,954, filed on Apr. 18, 2017.
(60) Provisional application No. 62/371,230, filed on Aug. 5, 2016.
(60) Provisional application No. 62/413,974, filed on Oct. 27, 2016.
(60) Provisional application No. 62/371,230, filed on Aug. 5, 2016.
(60) Provisional application No. 62/413,974, filed on Oct. 27, 2016.
(60) Provisional application No. 62/266,002, filed on Dec. 11, 2015.
(60) Provisional application No. 62/533,632, filed on Jul. 17, 2017.
(60) Provisional application No. 62/266,002, filed on Dec. 11, 2015.
(60) Provisional application No. 62/266,002, filed on Dec. 11, 2015.
(60) Provisional application No. 62/563,045, filed on Sep. 25, 2017.
(60) Provisional application No. 62/574,222, filed on Oct. 19, 2017.
(60) Provisional application No. 62/620,985, filed on Jan. 23, 2018.
(60) Provisional application No. 62/371,230, filed on Aug. 5, 2016.
(60) Provisional application No. 62/413,974, filed on Oct. 27, 2016.
(60) Provisional application No. 62/371,230, filed on Aug. 5, 2016.
(60) Provisional application No. 62/413,974, filed on Oct. 27, 2016.
(60) Provisional application No. 62/574,222, filed on Oct. 19, 2017.
(60) Provisional application No. 62/266,002, filed on Dec. 11, 2015.
(60) Provisional application No. 62/620,985, filed on Jan. 23, 2018.

(30) Foreign Application Priority Data
Aug. 26, 2014 (TW) 103129359
Dec. 22, 2014 (TW) 103144744
Jul. 18, 2016 (TW) 105122567
Sep. 26, 2016 (TW) 105214737
Nov. 4, 2016 (CN) 105135846
Mar. 17, 2017 (CN) 201720260844.9
Jun. 29, 2017 (TW) 106121692
Jul. 25, 2017 (CN) 201710612541.3
Aug. 8, 2017 (TW) 106126793
Sep. 20, 2017 (CN) 201710852899.3
Dec. 5, 2017 (CN) 201711271306.0
Jan. 7, 2018 (CN) 201820021470.X
Mar. 1, 2018 (CN) 201820286719.X
Apr. 24, 2018 (CN) 201820588432.2
Apr. 18, 2019 (CN) 201920526854.1
Dec. 17, 2019 (TW) 108216783

(51) Int. Cl. H01L 27/146

(52) U.S. Cl. CPC H01L 27/14627; H01L 27/14618; A61B 5/1172; H01L 27/14623; H01L 27/14678; H01L 27/1462

(57) ABSTRACT

An image capture device including an image sensor and a light collimator is provided. The light collimator is located on the image sensor and includes a light channel layer, a plurality of micro lenses and a wall structure. The plurality of micro lenses are disposed on the light channel layer, and the plurality of micro lenses and the image sensor are located on opposite sides of the light channel layer, respectively. The wall structure is disposed on the light channel layer and located at a periphery of the plurality of micro lenses, wherein a height of the wall structure is greater than a height of each of the plurality of micro lenses.

### CROSS-REFERENCE TO RELATED APPLICATION

This application is a continuation-in-part application of and claims the priority benefit of U.S. application Ser. No. 16/571,207, filed on Sep. 16, 2019. This application also claims the priority benefits of U.S. provisional application Ser. No. 62/906,103, filed on Sep. 26, 2019, U.S. provisional application Ser. No. 62/895,034, filed on Sep. 3, 2019, U.S. provisional application Ser. No. 63/003,929, filed on Apr. 2, 2020 and Taiwan application serial no. 108216783, filed on Dec. 17, 2019. The entirety of each of the above-mentioned applications is hereby incorporated by reference herein and made a part of this specification.

### BACKGROUND

### Field of the Invention

The disclosure relates to an electro-optical device and more particularly, to an image capture device.

### Description of Related Art

Types of biometrics include face, voice, iris, retina, vein, palm print and fingerprint identifications. According to different sensing methods, biological feature identification devices may be categorized into an optical type, a capacitive type, an ultrasonic type and a thermal-sensing type. Generally, an optical type feature identification device may include a light source, a light guide element and a sensor. A light beam emitted from the light source is irradiated on a test object which is pressed on the light guide element, and the sensor receives the light beam reflected by the test object for biological feature identification.

Taking the fingerprint identification as an example, when a finger is pressed on the light guide element, a ridge portion of the fingerprint contacts the light guide element, while a valley portion of the fingerprint does not the light guide element. Thus, the ridge portion of the fingerprint destroys total reflection of the light beam inside the light guide element, such that the sensor may obtain dark fringes corresponding to the ridge portion. In the meantime, the valley portion of the fingerprint does not destroy the total reflection of the light beam inside the light guide element, such that the sensor may obtain bright fringes corresponding to the valley portion. In this way, the light beam corresponding to the ridge portion and the valley portion of the fingerprint forms a bright and dark striped pattern on a light receiving surface of the sensor. The user's identity can then be identified by calculating information corresponding to a fingerprint image using an algorithm.

During an imaging process of the sensor, the light beam reflected by the fingerprint is easily scattered and transmitted to the sensor, such that a crosstalk is generated. This crosstalk may reduce a contrast ratio between a dark-fringe region and a bright-fringe region of the fingerprint pattern, which results in poor imaging quality and affects identification accuracy. Even though there are already techniques for improving the imaging quality, it is difficult for the techniques at the current stage to effectively improve the issue of crosstalk.

### SUMMARY

The invention provides an image capture device having a preferable identification capability.

An image capture device of the disclosure includes an image sensor and a light collimator. The light collimator is located on the image sensor and includes a light channel layer, a plurality of micro lenses and a wall structure. The plurality of micro lenses are disposed on the light channel layer, and the plurality of micro lenses and the image sensor are located on opposite sides of the light channel layer, respectively. The wall structure is disposed on the light channel layer and located at a periphery of the plurality of micro lenses, wherein a height of the wall structure is greater than a height of each of the plurality of micro lenses.

To sum up, in the embodiments of the disclosure, the light is collimated by the light collimator to improve the issue of crosstalk, such that the image capture device can have a preferable identification capability. In addition, the wall structure having the height higher than that of each of micro lenses are disposed at the periphery of the plurality of micro lenses, which can prevent the plurality of micro lenses from being damaged by scratches caused by accidental touches and facilitate subsequent assembly.

Several exemplary embodiments accompanied with figures are described in detail below to further describe the disclosure in details.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1A is a schematic cross-sectional diagram illustrating an image capture device according to an embodiment of the disclosure.

FIG. 1B illustrates a schematic top-view diagram of a light collimator depicted in FIG. 1A.

FIG. 2 through FIG. 8, FIG. 9A, FIG. 10 and FIG. 11 are schematic cross-sectional diagrams respectively illustrating image capture devices according to other embodiments of the disclosure.

FIG. 9B and FIG. 9C are schematic top-view diagrams respectively illustrating a light channel layer and an image sensor depicted in FIG. 9A.

### DESCRIPTION OF EMBODIMENTS

In the context hereinafter, wordings used to indicate directions, such as “up,” “down,” “front,” “back,” “left,” and “right”, merely refer to directions in the accompanying drawings. Thus, the language is used for describing the directions, but not intended to limit the scope of the disclosure.

In the accompanying drawings, the drawings illustrate the general features of the methods, structures, or materials used in the particular embodiments. Nevertheless, these drawings should not be construed as defining or limiting the scope or nature of what is covered by these embodiments. For instance, the relative thicknesses and locations of various film layers, regions, or structures may be reduced or enlarged for clarity.

In the embodiments below, the same or similar elements will be designated by the same or similar reference numerals, and descriptions thereof will be omitted. In addition, the features of different embodiments may be combined with each other when they are not in conflict, and simple equivalent changes and modifications made according to the specification or the claims are still within the scope of the disclosure.

Terms such as “first” and “second” mentioned throughout the specification or the claims of this application are only for naming the names of the elements or distinguishing different embodiments or scopes and are not intended to limit the upper limit or the lower limit of the number of the elements nor intended to limit manufacturing sequences or disposition sequences of the elements. In addition, an element/film layer disposed on another element/film layer may cover scenarios that the element/film layer is directly disposed on (or above) the other element/film layer and that the two element/film layer are in direct contact with each other, as well as scenarios that the element/film layer is indirectly disposed on (or above) the other element/film layer and that one or more elements/film layers exist between the two elements/film layers. For example, any two adjacent elements, any two adjacent film layers or adjacent element and film layer, if needed, may be fixed to each other through an adhesive layer (not shown) or a fixing mechanism (e.g., a screw or a locking member which is not shown), and the description will not be repeated hereinafter.

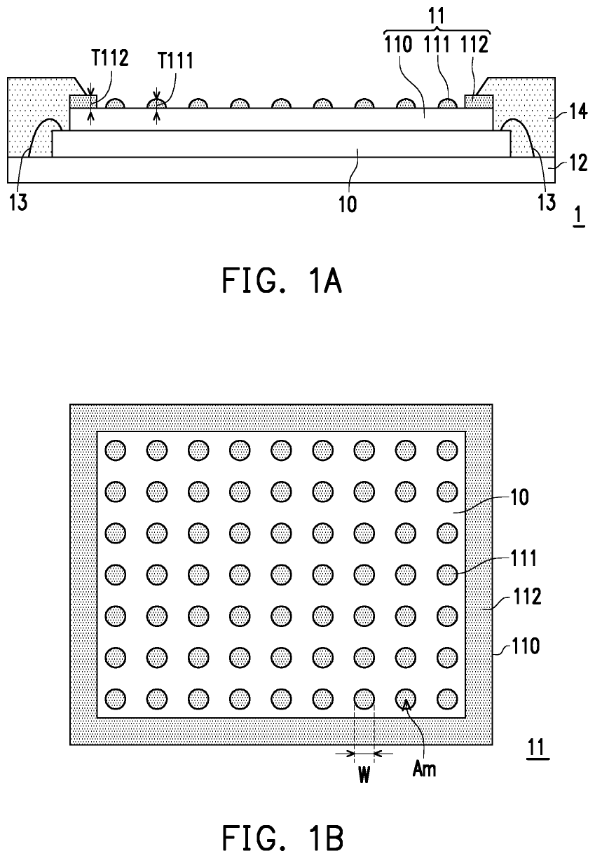

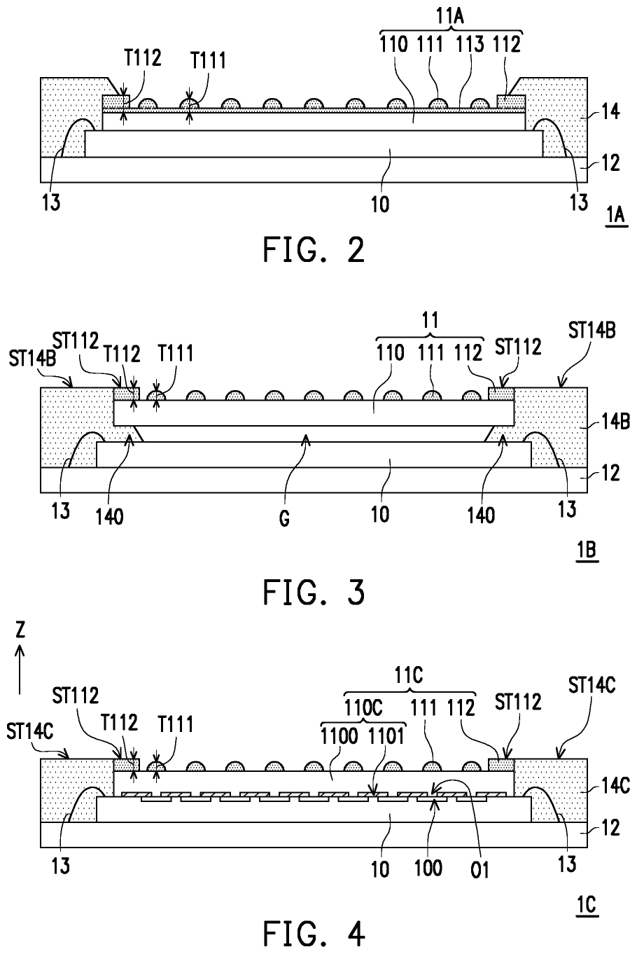

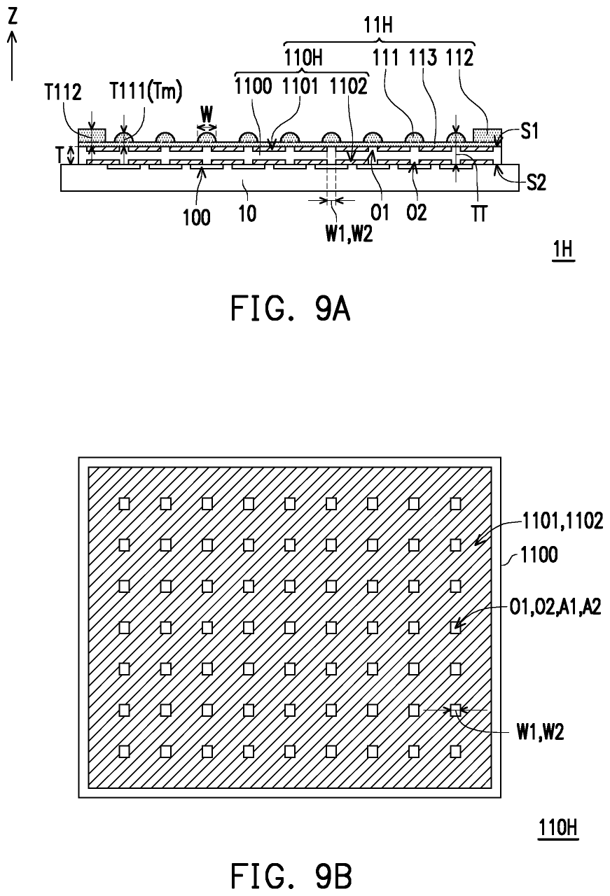

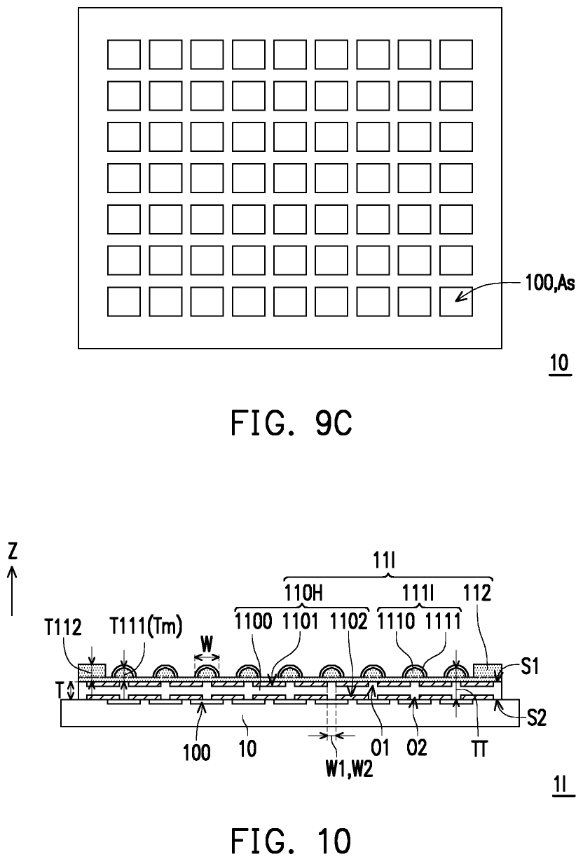

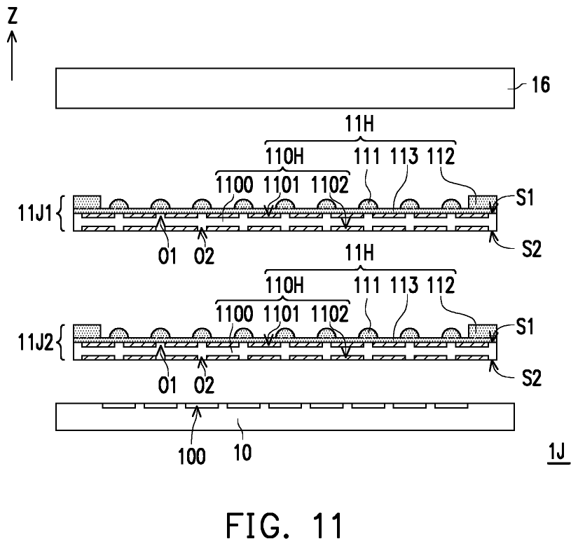

FIG. 1A is a schematic cross-sectional diagram illustrating an image capture device according to an embodiment of the disclosure. FIG. 1B illustrates a schematic top-view diagram of a light collimator depicted in FIG. 1A. FIG. 2 through FIG. 8, FIG. 9A, FIG. 10 and FIG. 11 are schematic cross-sectional diagrams respectively illustrating image capture devices according to other embodiments of the disclosure. FIG. 9B and FIG. 9C are schematic top-view diagrams respectively illustrating a light channel layer and an image sensor depicted in FIG. 9A.

In any embodiment of the disclosure, an image capture apparatus may be used in an environment medium. The environment medium may include air, water or other types of media. The image capture device is adapted to capture an image of a biological feature of a test object (not shown). For example, the test object may be a finger, a palm, a wrist or an eye-ball of a user, and a biological feature corresponding thereto may be a fingerprint, a palm print, a vein, a pupil or an iris, but the disclosure is not limited thereto.

Referring to FIG. 1A and FIG. 1B, an image capture device 1 includes an image sensor 10 and a light collimator 11. In the present embodiment, the image capture device 1 may further selectively include a circuit board 12, a plurality of metal lines 13 and an encapsulation layer 14, but the disclosure is not limited thereto.

The image sensor 10 is adapted to receive a light beam (which is a light beam with biological feature information) reflected by the test object. For example, the image sensor 10 may include a charge coupled device (CCD), a complementary metal oxide semiconductor (CMOS) or other adaptive types of image sensing elements.

The light collimator 11 is located on the image sensor 10. The light collimator 11 is adapted to collimate the light beam reflected by the test object to improve an issue of crosstalk, such that the image capture device 1 may have a preferable identification capability. The light collimator 11 may include a light channel layer 110, a plurality of micro lenses 111 and a wall structure 112.

The light channel layer 110 is adapted to collimate the light beam reflected by the test object. For example, the light channel layer 110 may include a combination of a light-shielding layer and a light-transmitting layer, a plurality of optical fibers, a plurality of pinholes, a grating or other adaptive light collimating elements.

The plurality of micro lenses 111 are disposed on the light channel layer 110, and the plurality of micro lenses 111 and the image sensor 10 are located on opposite sides of the light channel layer 110, respectively. The plurality of micro lenses 111 are adapted to converge light beams for allowing more light beams to pass through the light channel layer 110 and to be received by the image sensor 10.

In the present embodiment, referring to FIG. 1A, a cross-sectional shape of each of the plurality of micro lenses 111 is a hemispherical shape. Nevertheless, the cross-sectional shape of each of the plurality of micro lenses 111 may also be changed as other shapes based on demands, and the hemispherical shape is not limited to a half of a sphere. In addition, referring to FIG. 1B, a projection shape of each of the plurality of micro lenses 111 on the light channel layer 110 may be a circular shape. Nevertheless, the projection shape of each of the plurality of micro lenses 111 may also be changed as other shapes based on demands, for example, a quadrilateral shape or a polygonal shape.

The wall structure 112 is disposed on the light channel layer 110 and located at a periphery of the plurality of micro lenses 111. As illustrated in FIG. 1A, the wall structure 112 and the plurality of micro lenses 111 are disposed on a same surface of the light channel layer 110. In addition, a height T112 of the wall structure 112 is greater than a height T111 of each of the plurality of micro lenses 111. Based on the surface of the light channel layer 110 which bears the wall structure 112 and the plurality of micro lenses 111, the height T112 of the wall structure 112 may be a maximum distance between a bottom surface (i.e., a surface of the wall structure 112 in contact with the light channel layer 110) and a top surface of the wall structure 112. In the same way, based on the surface of the light channel layer 110 which bears the wall structure 112 and the plurality of micro lenses 111, the height T111 of each of the plurality of micro lenses 111 may be a maximum distance between a bottom surface (i.e., a surface of any one of the micro lens 111 in contact with the light channel layer 110) and a top surface of the micro lens 111. The wall structure 112 having the height higher than that of each of micro lenses 111 is disposed at the periphery of the plurality of micro lenses 111, which may prevent the plurality of micro lenses 111 from being damaged by scratches caused by accidental touches and facilitate subsequent assembly.

In an embodiment, the wall structure 112 and the plurality of micro lenses 111 may be integrally formed, so as to simplify the number of processes and reduce a process time, but the disclosure is not limited thereto. In another embodiment, the wall structure 112 and the plurality of micro lenses 111 may be respectively formed on the light channel layer 110, and the wall structure 112 and the plurality of micro lenses 111 may be made of the same material or different materials.

In the present embodiment, as illustrated in FIG. 1A, a cross-sectional shape of the wall structure 112 may be a rectangular shape. In addition, as illustrated in FIG. 1B, a projection shape of the wall structure 112 on the light channel layer 110 may be a frame shape, and the wall structure 112 may surround the plurality of micro lenses 111. Nevertheless, the cross-sectional shape and the projection shape of the wall structure 112 or a relative disposition relationship between the wall structure 112 and the plurality of micro lenses 111 may vary with demands, without being limited to those illustrated in FIG. 1A and FIG. 1B. For example, the cross-sectional shape of the wall structure 112 may also be a square shape, a trapezoidal shape or other polygonal shapes.

In an embodiment, the wall structure 112 may include a plurality of pseudo-micro lenses (not shown). The plurality of pseudo-micro lenses are disposed at the periphery of the plurality of micro lenses 111, and a height of each of the plurality of pseudo-micro lenses is greater than the height of each of the plurality of micro lenses 111, thereby protecting the plurality of micro lenses 111. Since the plurality of pseudo-micro lenses mainly functions as protecting the plurality of micro lenses 111, the design of parameters (e.g., a curvature radius, a refractive index and so on) of the plurality of pseudo-micro lenses may not be particularly limited herein. For example, the plurality of pseudo-micro lenses and the plurality of micro lenses 111 may be made of the same material or different materials, have the same or different cross-sectional shapes and/or have the same or different projection shapes.

The circuit board 12 is adapted to bear the image sensor 10, and the image sensor 10 is disposed on the circuit board 12. The circuit board 12 may be a printed circuit board (PCB) or a flexible printed circuit (FPC) or a substrate on which circuit layers are formed.

The plurality of metal lines 13 electrically connects the image sensor 10 and the circuit board 12. For example, the plurality of metal lines 13 may be connected between pads (not shown) of the image sensor 10 and pads (not shown) of the circuit board 12 by a wire bonding process.

The encapsulation layer 14 encapsulates the plurality of metal lines 13 and fixes the image sensor 10 and the light collimator 11 on the circuit board 12. The encapsulation layer 14 may be formed by using a molding compound in the art, but the disclosure is not limited thereto. In the present embodiment, the image sensor 10, the light collimator 11 and the plurality of metal lines 13 are first formed on the circuit board 12, and then, the image sensor 10, the light collimator 11 and the plurality of metal lines 13 are fixed by the encapsulation layer 14. Thus, the encapsulation layer 14 covers an edge portion of the light collimator 11, e.g., an outer edge covering the wall structure 112.

The formation of the encapsulation layer 14 may facilitate preferable electrical performance (e.g., preventing oxidization of the plurality of metal lines 13 or poor bonding between the plurality of metal lines 13 and the circuit board 12) and improving a mechanical strength of the image sensor 10 (e.g., preventing the image sensor 10 from being easily broken due to being thinned). In this way, it facilitates satisfying demand for thinning and improvement of yield, without significantly increasing the process time. In addition, the encapsulation layer 14 may further shield a light source disposed on a side or other stray light from entering a transparent substrate 1100, so as to prevent the issue of crosstalk caused by the stray light.

Based on different design demands, the image capture device 1 may further include other elements. For example, the image capture device 1 may further include a cover plate (not shown), a light filter layer (not shown), a middle frame (not shown), an adhesive layer (not shown), a fixing mechanism (not shown), a light source (not shown) or a combination of at least two of the aforementioned elements. The embodiments below may also be improved in the same way, and the descriptions will not be repeated hereinafter.

Referring to FIG. 2, an image capture device 1A and the image capture device 1 illustrated in FIG. 1A are different from each other mainly in a light collimator 11A of the image capture device 1A further including a base layer 113. The plurality of micro lenses 111 and the wall structure 112 are disposed on the base layer 113 and in contact with the base layer 113. For example, the base layer 113, the wall structure 112 and the plurality of micro lenses 111 may be integrally formed, and the base layer 113, the wall structure 112 and the plurality of micro lenses 111 may be formed by means of imprinting or molding. In the presence of the base layer 113, the height T112 of the wall structure 112 may be a maximum distance between a bottom surface of the base layer 113 (i.e., a surface of the base layer 113 in contact with the light channel layer 110) and atop surface of the wall structure 112, and the height T111 of each of the plurality of micro lenses 111 may be a maximum distance between the bottom surface of the base layer 113 and a top surface of any one of the micro lenses 111. In addition, in any embodiment of the application, the cross-sectional shape of the wall structure 112 may also be a stepped shape (e.g., an L shape and a shape laterally reversed thereto) and served as extension of the wall structure 112. The plurality of micro lenses 111 may be disposed between two step-shaped wall structures 112.

Referring to FIG. 3, an image capture device 1B and the image capture device 1 illustrated in FIG. 1A are different from each other mainly in the follows. In the image capture device 1B, an encapsulation layer 14B has a supporting portion 140. The supporting portion 140 is located between the image sensor 10 and the light collimator 11, and the light collimator 11 is supported by the supporting portion 140. Furthermore, in the present embodiment, the image sensor 10 and the plurality of metal lines 13 are first formed on the circuit board 12, and then, the image sensor 10 and the plurality of metal lines 13 are fixed by the encapsulation layer 14B. Thereafter, the light collimator 11 is disposed on the supporting portion 140 of the encapsulation layer 14B. In this way, a gap G exists between the light collimator 11 and the image sensor 10. An optical transmission medium in the gap G may be air, but the disclosure is not limited thereto.

In the present embodiment, a top surface ST112 of the wall structure 112 is aligned with or almost aligned with a top surface ST14B of the encapsulation layer 14B. In other words, the top surface ST14B of the encapsulation layer 14B may also be taller than the plurality of micro lenses 111. In this architecture, the wall structure 112 and the encapsulation layer 14B may collectively protect the plurality of micro lenses 111. In addition, the top surface ST112 of the wall structure 112 and the top surface ST14B of the encapsulation layer 14B may form a planar surface adapted to bear other elements (e.g., a cover plate, a light filter layer, a middle frame or the like).

Referring to FIG. 4, an image capture device 1C and the image capture device 1 illustrated in FIG. 1A are different from each other mainly in the follows. In the image capture device 1C, a light channel layer 110C of a light collimator 11C includes a transparent substrate 1100 and a first light-shielding layer 1101.

The transparent substrate 1100 is adapted to allow the light beam to pass through and may be made of any transparent material, and the material of the transparent substrate 1100 is not particularly limited herein. The light-shielding layer 1101 is disposed on a side of the transparent substrate 1100. For example, the first light-shielding layer 1101 may be disposed on a side of the transparent substrate 1100 which faces the image sensor 10 or on a side of the transparent substrate 1100 which is far away from the image sensor 10. Alternatively, the light-shielding layer 1101 may be formed in the transparent substrate 1100.

The first light-shielding layer 1101 is adapted to absorb the light beam to mitigate the affection caused by the stray light to a biological feature identification result. The first light-shielding layer 1101 may be fabricated by using any light-absorption material, and the material of the first light-shielding layer 1101 is not particularly limited herein. The first light-shielding layer 1101 has a plurality of first openings O1. The plurality of first openings O1, the plurality of micro lenses 111 and a plurality of image sensing elements 100 of the image sensor 10 overlap with one another in a thickness direction Z of the image capture device 1C.

The top surface ST112 of the wall structure 112 is aligned with a top surface ST14C of an encapsulation layer 14C, and the encapsulation layer 14C does not cover a top portion of the light collimator 11C. For example, the encapsulation layer 14C does not cover the wall structure 112 of the light collimator 11C.

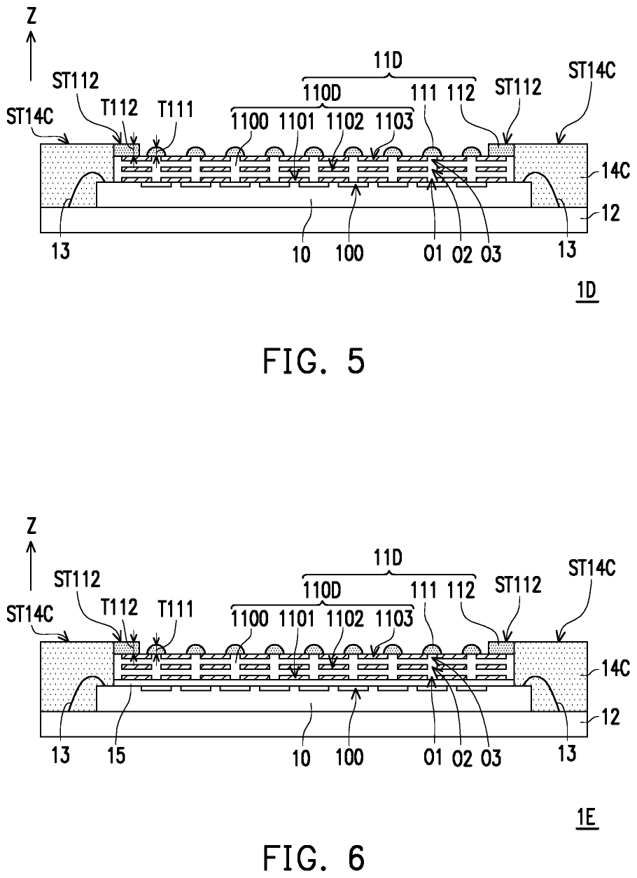

Referring to FIG. 5, an image capture device 1D and the image capture device 1C illustrated in FIG. 4 are different from each other mainly in the follows. In the image capture device 1D, a light channel layer 110D of a light collimator 11D further includes a second light-shielding layer 1102 and a third light-shielding layer 1103. The third light-shielding layer 1103 is disposed on a side of the transparent substrate 1100 which is far away from the image sensor 10, the third light-shielding layer 1103 is formed in the transparent substrate 1100, and the second light-shielding layer 1102 is located between the first light-shielding layer 1101 and the third light-shielding layer 1103. The second light-shielding layer 1102 and the third light-shielding layer 1103 may be fabricated by using any light-absorption material, and the materials of the second light-shielding layer 1102 and the third light-shielding layer 1103 are not particularly limited herein. The second light-shielding layer 1102 has a plurality of second openings O2, and the third light-shielding layer 1103 has a plurality of third openings O3. The plurality of first openings O1, the plurality of second openings O2, the third openings O3, the plurality of micro lenses 111 and the image sensing elements 100 of the image sensor 10 overlap with one another in a thickness direction Z of the image capture device 1D.

In the present embodiment, the plurality of first openings O1, the plurality of second openings O2 and the plurality of third openings O3 have the same size. Nevertheless, the sizes of the openings of each light-shielding layer or the numbers of the light-shielding layers in the light channel layer 110D may be designed based on demands, without being limited to those illustrated in FIG. 5.

Referring to FIG. 6, an image capture device 1E and the image capture device 1E illustrated in FIG. 5 are different from each other mainly in the image capture device 1E further including a light filter layer 15. The light filter layer 15 is adapted to filter the stray light. For example, when biological feature identification is performed by using visible light, the light filter layer 15 may be used to filter the light (e.g., infrared light) having a wave band other than that of the visible light. On the other hand, when the biological feature identification is performed by using the infrared light, the light filter layer 15 may be used to filter the light having a wave band other than that of the infrared light. In the present embodiment, the light filter layer 15 is located between the image sensor 10 and the light collimator 11D. In another embodiment, the light filter layer 15 may be disposed above the top surface ST112 of the wall structure 112 and the top surface ST14C of the encapsulation layer 14C. Or, alternatively, the light filter layer 15 may be disposed between the image sensor 10 and the light collimator 11D and above the top surface ST112 of the wall structure 112 and the top surface ST14C of the encapsulation layer 14C, simultaneously.

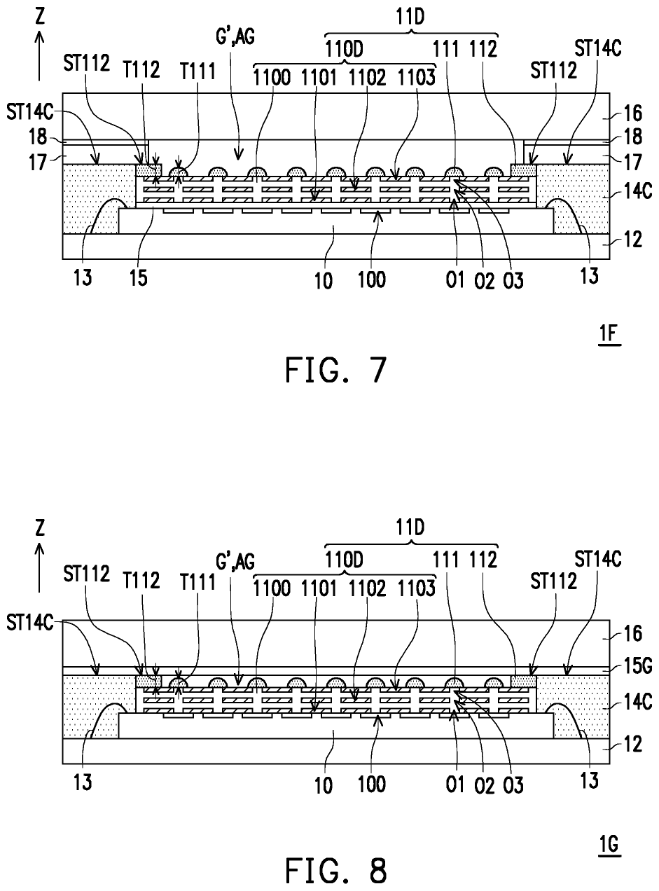

Referring to FIG. 7, an image capture device 1F and the image capture device 1E illustrated in FIG. 6 are different from each other mainly in the follows. The image capture device 1F further includes a cover plate 16, a middle frame 17 and a shading buffer layer 18 (e.g., shading foam).

The cover plate 16 is located above the encapsulation layer 14C, wherein the light collimator 11D is located between the cover plate 16 and the image sensor 10. A surface of the cover plate 16 which is far away from the light collimator 11D may be a pressing surface of the test object, i.e., the test object is pressed on the surface of the cover plate 16 which is far away from the light collimator 11D for performing the biological feature identification.

The cover plate 16 is adapted to protect elements, such as the light collimator 11D and the image sensor 10, disposed thereunder. For example, the cover plate 16 may include a transparent substrate, a transparent film, a transparent display panel, a transparent touch panel, a transparent touch display panel or a combination of at least two of the aforementioned elements. The transparent display panel may be a transparent thin film transistor liquid crystal display (TFT-LCD) panel, a micro light emitting diode (micro LED) display panel or an organic light emitting diode (OLED) display panel, but the disclosure is not limited thereto. The difference between the transparent touch display panel and transparent display panel lies in the transparent touch display panel further including a touch function. For example, transparent touch display panel may include a touch electrode, but the disclosure is not limited thereto.

A part of an image light beam (e.g., visible light) provided by the transparent display panel or the transparent touch display panel may be used in the biological feature identification, but the disclosure is not limited thereto. In an embodiment, the image capture device 1F may further include a light source (not shown) configured to provide a light beam for the biological feature identification. A wave length of the light beam provided by the light source may be different from a wave length of the image light beam (a visible light wave length). For example, the light source may be an invisible light source, e.g., an infrared light source, but the disclosure is not limited thereto. In addition, the light source may be disposed outside the transparent display panel or the transparent touch display panel, or alternatively, integrated in the transparent display panel or the transparent touch display panel.

The middle frame 17 is located between the encapsulation layer 14C and the cover plate 16, and the middle frame 17 may be disposed on the top surface ST112 of the wall structure 112 and the top surface ST14C of the encapsulation layer 14C. In another embodiment, the middle frame 17 may be disposed on the top surface ST14C of the encapsulation layer 14C, without overlapping the wall structure 112 in a thickness direction Z of the image capture device 1F.

The middle frame 17 and the cover plate 16 are bonded together by the shading buffer layer 18. In the present embodiment, a projection shape of the shading buffer layer 18 on the cover plate 16 is a frame shape, and the shading buffer layer 18 may not overlap the plurality of micro lenses 111 in the thickness direction Z of the image capture device 1F. Namely, the shading buffer layer 18 is not filled in a gap G′ between the cover plate 16 and the plurality of micro lenses 111, such that an air gap AG exists between the cover plate 16 and the plurality of micro lenses 111.

The light filter layer 15 is located between the image sensor 10 and the cover plate 16. For example, the light filter layer 15 may be located between the image sensor 10 and the light collimator 11D or between the light collimator 11D and the cover plate 16.

Referring to FIG. 8, an image capture device 1G and the image capture device 1F illustrated in FIG. 7 are different from each other mainly in the follows. The middle frame 17 and the light filter layer 15 as illustrated in FIG. 7 are omitted in the image capture device 1G. In addition, the image capture device 1G further includes a light filter layer 15G located between the light collimator 11D and the cover plate 16. The description related to the light filter layer 15G may refer to the description related the light filter layer 15 and will not be repeated hereinafter. In another embodiment, the light filter layer 15G is omitted, and the cover plate 16 is attached to the encapsulation layer 14C via an adhesive layer (e.g., the shading buffer layer 18 shown in FIG. 7).

Referring to FIG. 9A to FIG. 9C, an image capture device 1H and the image capture device 1A illustrated in FIG. 2 are different from each other mainly in the follows. The circuit board 12, the plurality of metal lines 13 and the encapsulation layer 14 as illustrated in FIG. 2 are omitted in the image capture device 1H. Nevertheless, in an embodiment, the image capture device 1H may further include the circuit board 12, the plurality of metal lines 13 and the encapsulation layer 14 as illustrated in FIG. 2. Or, alternatively, the image capture device 1H may further include the circuit board 12, the plurality of metal lines 13 and the encapsulation layer 14B as illustrated in FIG. 3.

In the image capture device 1H, a light channel layer 110H of a light collimator 11H includes a transparent substrate 1100, a first light-shielding layer 1101 and a second light-shielding layer 1102. The transparent substrate 1100 has a first surface S1 and a second surface S2. The first surface S1 is located between the plurality of micro lenses 111 and the second surface S2. The first light-shielding layer 1101 is disposed on the first surface S1 and has a plurality of first openings O1. The second light-shielding layer 1102 is disposed on the second surface S2 and has a plurality of second openings O2. The plurality of first openings O1, the plurality of second openings O2, the plurality of micro lenses 111 and the image sensing elements 100 overlap with one another in a thickness direction Z of the image capture device 1H.

An area of each of the plurality of image sensing elements 100 is As. A projected area of each of the plurality of micro lenses 111 is Am (referring to FIG. B). An area of each of the plurality of first openings O1 is A1. An area of each of the plurality of second openings O2 is A2. The image capture device satisfies at least one of A1≤A2<Am, A1≤A2<As, and A1≤A2<Am<As. Alternatively, the image capture device satisfies at least one of A2≤A1<Am, A2≤A1<As, and A2≤A1<Am<As. By satisfying the above design of the areas, the image capture device 1H may have preferable imaging quality, such that the image capture device 1H may have a preferable identification capability.

Table 1 below shows some examples. The unit of “A1”, “A2”, “Am”, or “As” is μm². The unit of “W”, “Tm”, or “T” is μm. In table 1, “E” refers to the evaluation factor, wherein E=avg(low)/avg(high). In the equation, avg(low) refers to average value of low amplitude signal, and avg(high) refers to average value of high amplitude signal. When the evaluation factor is equal to or lower than 30%, the image captured by the image capture device is not recognizable.

TABLE 1

| Example | A1 | A2 | Am | As | W | Tm | T | E (%) |
|---|---|---|---|---|---|---|---|---|
| 1 | 78.5 | 78.5 | 490.62 | 625 | 25 | 4.387 | 30 | 20.2 |
| 2 | 78.5 | 78.5 | 346.18 | 625 | 21 | 2.98 | 30 | 25 |
| 3 | 314 | 78.5 | 346.18 | 625 | 21 | 2.98 | 40 | 37.6 |
| 4 | 78.5 | 153.86 | 346.18 | 625 | 25 | 4.387 | 46 | 29.8 |
| 5 | 78.5 | 153.86 | 113.04 | 625 | 25 | 4.387 | 30 | 46.3 |

In the structure of FIG. 9A, the image capture device 1H satisfies T≤π[(W/2)²+Tm²)]/(2Tm) and may also have preferable imaging quality, such that the image capture device 1H may have the preferable identification capability. In the relational formula set forth above, T is a thickness of the transparent substrate 1100 (for example, a maximum thickness of the transparent substrate 1100), W is a width of each of the plurality of micro lenses 111 (for example, a maximum width of the projection shape of each of the plurality of micro lenses 111), and Tm is a width of each of the plurality of micro lenses 111 (for example, a maximum thickness of each of the plurality of micro lenses 111 (where Tm=T111)). In an embodiment, the image capture device 1H, if satisfying T≤π[(W/2)²+Tm²)]/(4Tm), may have preferable imaging quality, such that the image capture device 1H may have the preferable identification capability.

In the present embodiment, a width W1 of each of the plurality of first openings O1 and a width W2 of each of the plurality of second openings O2 satisfy, for example, 2 μm≤W1≤As, and 2 μm≤W2≤As.

According to the aforementioned design, the image capture device 1H may meet the demand for thinning. In an embodiment, a maximum thickness TT of a stack structure of the plurality of micro lenses 111 and the light channel layer 110H (including the base layer 113 if there is any) is less than 100 μm, which may be 80 μm, for example, but the disclosure is not limited thereto.

Referring to FIG. 10, an image capture device 1I and the image capture device 1H illustrated in FIG. 9A are different from each other mainly in that each of a plurality of micro lenses 111I of a light collimator 11I is a multi-layered structure. In the present embodiment, each of the micro lenses 111I includes a first layer 1110 and a second layer 1111. The first layer 1110 and the second layer 1111 may be made of different materials, and the materials of the first layer 1110 and the second layer 1111 may be selected based on demands and are not particularly limited herein. In other embodiments, each of the micro lenses 111I may include more layers. In addition, any embodiment of the disclosure may be improved in the same way, and the descriptions will not be repeated hereinafter.

Referring to FIG. 11, an image capture device 1J and the image capture device 1H illustrated in FIG. 9A are different from each other mainly in the follows. In the image capture device 1J, the image capture device 1J includes the image sensor 10, a light collimator 11J1 and the cover plate 16. For descriptive convenience, a light collimator 11J2 located between the light collimator 11J1 and the image sensor 10 may be referred to as an inner light collimator.

In the present embodiment, the light collimator 11J1 and the light collimator 11J2 may use the structure of the light collimator 1H illustrated in FIG. 9A, but the disclosure is not limited thereto. In another embodiment, the light collimator 11J1 and the light collimator 11J2 may use the structures of the light collimators of other embodiments. Or, alternatively, the light collimator 11J1 and the light collimator 11J2 may have different structures. For example, the wall structure 112 may be omitted from the light collimator 11J2. In other embodiments, the image capture device 1J may include multiple inner light collimators, and the multiple inner light collimators may be arranged between the image sensor 10 and the light collimator 11J1 along a thickness direction Z of the image capture device 11J. In addition, the plurality of micro lenses 111 of the inner light collimator (e.g., the light collimator 11J2) overlap the plurality of micro lenses 111 of the light collimator 11J1 in a stack direction of the light collimator 11J1 and the inner light collimator (e.g., the light collimator 11J2).

Based on the above, in the embodiments of the disclosure, the light is collimated by the light collimator to improve the issue of crosstalk, such that the image capture device can have a preferable identification capability. In addition, the wall structure having the height higher than that of each of micro lenses are disposed at the periphery of the plurality of micro lenses, which can prevent the plurality of micro lenses from being damaged by scratches caused by accidental touches and facilitate subsequent assembly.

In an embodiment, the wall structure and the plurality of micro lenses can be integrally formed so as to simplify the number of processes and reducing the process time. In an embodiment, the the formation of the encapsulation layer can maintain preferable electrical performance and improve the mechanical strength of the image sensor, so as to satisfy the demand for thinning and improve the yield. In an embodiment, the top surface of the wall structure and the top surface of the encapsulation layer can be aligned with each other to form the planar surface adapted to bear other elements. In an embodiment, the light filter layer can be disposed to filter the stray light. In an embodiment, the cover plate can be disposed to protect the elements thereunder. The cover plate can include the transparent substrate, the transparent film, the transparent display panel, the transparent touch panel, the transparent touch display panel or the combination of at least two of the aforementioned elements. In an embodiment, a part of the image light beam provided by the transparent display panel or the transparent touch display panel can be used for performing the biological feature identification, or alternatively, the image capture device can further include the light source configured to provide the light beam for performing the biological feature identification. In an embodiment, the imaging quality can be enhanced through the design of the areas of the light collimator and the image sensing elements. In an embodiment, the imaging quality can be enhanced through the design of the thickness of the transparent substrate and the thickness and the width of each of the micro lenses. In an embodiment, the image capture device can meet the demand for thinning. In an embodiment, each of the micro lenses can have the multi-layered structure based on demands. In an embodiment, the image capture device can have a plurality of light collimators (including the light collimators and the inner light collimators).

Although the invention has been disclosed by the above embodiments, they are not intended to limit the invention. It will be apparent to one of ordinary skill in the art that modifications and variations to the invention may be made without departing from the spirit and scope of the invention. Therefore, the scope of the invention will be defined by the appended claims.

What is claimed is:

1. An image capture device, comprising:
an image sensor; and
a light collimator, located on the image sensor, wherein the light collimator comprises:
a light channel layer;
a plurality of micro lenses, disposed on the light channel layer, and the plurality of micro lenses and the image sensor being located on opposite sides of the light channel layer, respectively; and
a wall structure, disposed on the light channel layer and located at a periphery of the plurality of micro lenses, wherein a height of the wall structure is greater than a height of each of the plurality of micro lenses.

2. The image capture device according to claim 1, wherein a projection shape of the wall structure on the light channel layer is a frame shape, and the wall structure surrounds the plurality of micro lenses.

3. The image capture device according to claim 1, wherein the wall structure comprises a plurality of pseudo-micro lenses, wherein a height of each of the plurality of pseudo-micro lenses is greater than the height of each of the plurality of micro lenses.

4. The image capture device according to claim 1, further comprising:
a circuit board, wherein the image sensor is disposed on the circuit board;
a plurality of metal lines, electrically connecting the image sensor and the circuit board; and
an encapsulation layer, encapsulating the plurality of metal lines and fixing the image sensor and the light collimator on the circuit board.

5. The image capture device according to claim 4, wherein the encapsulation layer covers an edge portion of the light collimator.

6. The image capture device according to claim 4, wherein the encapsulation layer has a supporting portion, the supporting portion is located between the image sensor and the light collimator, the light collimator is supported by the supporting portion, and a gap exists between the light collimator and the image sensor.

7. The image capture device according to claim 4, wherein a top surface of the wall structure is aligned with a top surface of the encapsulation layer.

8. The image capture device according to claim 4, further comprising:
a cover plate, located above the encapsulation layer, wherein the light collimator is located between the cover plate and the image sensor, and an air gap exists between the cover plate and the plurality of micro lenses.

9. The image capture device according to claim 8, wherein the cover plate comprises a transparent substrate, a transparent film, a transparent display panel, a transparent touch panel, a transparent touch display panel or a combination of at least two of the aforementioned elements.

10. The image capture device according to claim 8, further comprising:
a middle frame, located between the encapsulation layer and the cover plate.

11. The image capture device according to claim 8, further comprising:
a light filter layer, located between the image sensor and the cover plate.

12. The image capture device according to claim 1, wherein the wall structure and the plurality of micro lenses are integrally formed.

13. The image capture device according to claim 1, wherein each of the micro lenses is a multi-layered structure.

14. The image capture device according to claim 1, wherein the light channel layer comprises a combination of a light-shielding layer and a light-transmitting layer, a plurality of optical fibers, a plurality of pinholes or a grating.

15. The image capture device according to claim 1, wherein the image sensor comprises a plurality of image sensing elements, and the light channel layer comprises:
a transparent substrate, having a first surface and a second surface, and the first surface being located between the plurality of micro lenses and the second surface;
a first light-shielding layer, disposed on the first surface and having a plurality of first openings; and
a second light-shielding layer, disposed on the second surface and having a plurality of second openings,
wherein the plurality of first openings, the plurality of second openings, the plurality of micro lenses and the plurality of image sensing elements overlap with one another, and
wherein an area of each of the plurality of image sensing elements is As, a projected area of each of the plurality of micro lenses is Am, an area of each of the plurality of first openings is A1, an area of each of the plurality of second openings is A2, and the image capture device satisfies A1≤A2<Am<As.

16. The image capture device according to claim 15, wherein a thickness of the transparent substrate is T, a width of each of the plurality of micro lenses is W, a thickness of each of the plurality of micro lenses is Tm, and the image capture device satisfies T≤π[(W/2)²+Tm²)]/(2Tm).

17. The image capture device according to claim 15, wherein a maximum thickness of a stack structure of the plurality of micro lenses and the light channel layer is less than 100 μm.

18. The image capture device according to claim 1, further comprising:
an inner light collimator, located between the light collimator and the image sensor and comprising:
a light channel layer; and
a plurality of micro lenses, disposed on the light channel layer of the inner light collimator, and the plurality of micro lenses and the image sensor of the inner light collimator being located on opposite sides of the light channel layer of the inner light collimator, respectively, wherein the plurality of micro lenses of the inner light collimator overlap the plurality of micro lenses of the light collimator in a stack direction of the light collimator and the inner light collimator.

19. The image capture device according to claim 18, wherein the inner light collimator further comprises:
a wall structure, disposed on the light channel layer of the inner light collimator and located at a periphery of the plurality of micro lenses of the inner light collimator, wherein a height of the wall structure of the inner light collimator is greater than a height of each of the plurality of micro lenses of the inner light collimator.
